(19) United States
(12) Plant Patent Application Publication
(10) Pub. No.: US 2015/0282412 P1
(43) Pub. Date: Oct. 1, 2015

(54) Adenathos plant named 'AVIV'

(50) ;

(71) Applicant: Nitzan Nir, Kfar Hess (IL)

(72) Inventor: Nitzan Nir, Kfar Hess (IL)

(21) Appl. No.: 13/999,886

(22) Filed: Mar. 31, 2014

(51) Int. Cl. A01H 5/00

(52) U.S. Cl. PLT/373

(57) ABSTRACT

A new and distinct Adenanthos cultivar named ‘AVIV’ is disclosed, characterized by unique branch structure and prostrate plant habit. The new variety is a Adenanthos, normally used for ornamental foliage, or landscape purposes.

### LATIN NAME OF THE GENUS AND SPECIES

Adenanthos sericeus

### VARIETY DENOMINATION

‘AVIV’

### BACKGROUND OF THE INVENTION

The new Adenanthos cultivar is a product of open pollination. The new variety was discovered as a seedling growing in a field of various Adenanthos sericeus varieties by the inventor, Nitzan Nir, in Kfar Hess, Israel. The new variety was selected during 2008, at a commercial nursery in Kfar Hess, Israel. The exact parents are unknown, and believed to be unnamed varieties of Adenanthos.

Asexual reproduction of the new cultivar has been performed by vegetative cuttings. This was first performed at a commercial nursery in Kfar Hess, Israel in 2008 and has shown that the unique features of this cultivar are stable and reproduced true to type in 6 successive generations.

### SUMMARY OF THE INVENTION

The cultivar ‘AVIV’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘AVIV’. These characteristics in combination distinguish ‘AVIV’ as a new and distinct Adenanthos cultivar:

1. Branch structure.

2. Prostrate plant form.

### PARENT COMPARISON

The parents are unknown to the inventor.

### COMMERCIAL COMPARISON

Plants of the new cultivar ‘AVIV’ are comparable to the unpatented commercial variety Adenanthos sericeus. ‘TAL’, application lodged concurrently. The two Adenanthos varieties are similar in most horticultural characteristics; however, the new variety ‘AVIV’ differs in the following characteristics;

1. Plants of ‘AVIV’ are prostrate, whereas plants of ‘TAL’ are erect.

Plants of the new cultivar ‘AVIV’ can also be compared to the unnamed, unpatented commercial variety Adenanthos sericeus. Varieties are similar in most horticultural characteristics; however ‘AVIV’ differs in the following characteristics;

1. Prostrate plant habit, compared to semi-erect plant habit.

### BRIEF DESCRIPTION OF THE PHOTOGRAPH

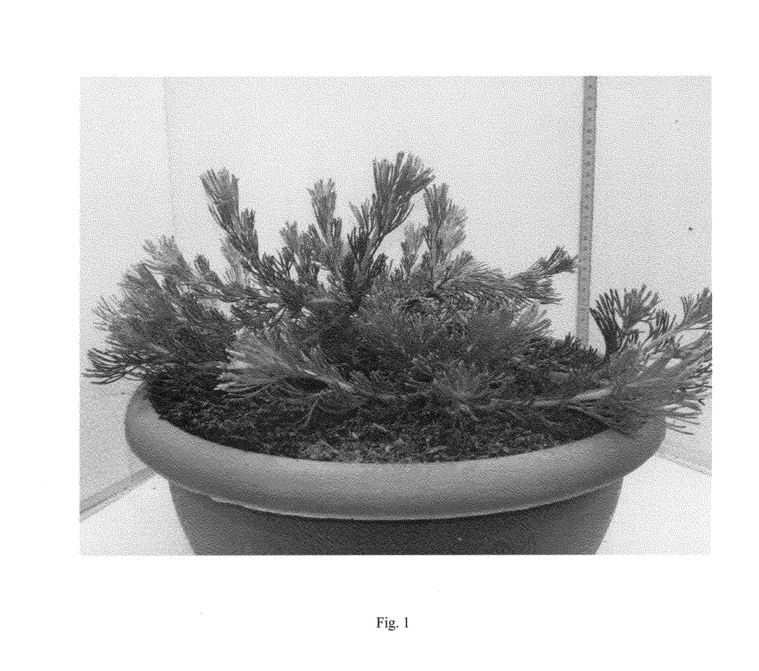

The accompanying photograph in FIG. 1 illustrates in full color a typical plant of ‘AVIV’ grown in a commercial greenhouse in Kfar, Israel. The plant is 4-5 months old from a rooted cutting, in a 25 cm pot. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007 except where general terms of ordinary dictionary significance are used. The following observations and measurements describe typical plant of ‘AVIV” grown in a commercial greenhouse in Kfar, Israel. The plant is at least 2 months old from a rooted cutting, in a 25 cm pot. The growing temperature ranged from 15° C. to 35° C. during the day and from 2° C. to 35° C. during the night. General light conditions are bright, normal sunlight. Measurements and numerical values represent averages of typical plant types. Chemical treatments included general fungicides and insecticides.
Botanical classification: Adenanthos sericeus ‘AVIV’
Propagation:
   Time to produce a rooted plantlet.—30 to 60 days at approximately 24° C.
   Root description.—Split root.
Plant:
   Growth habit.—Prostrate Shrub.
   Height.—20-40 cm to top of foliage.
   Plant spread.—200 cm.
   Branching characteristics.—Moderate branching from base.
   Length of primary lateral branches.—15 to 35 cm.
   Quantity Of primary lateral branches.—Approximately 12 to 18.
   Characteristics of primary lateral branches.—Form: Round in cross section Diameter: 0.2-0.6 cm. Color: Grey-green 194A RHS Texture: Hairy Strength: Flexible.
   Internode length.—0.2-5.0 cm. (measured in section of 20 cm from top).
Foliage:
   Leaf.—Arrangement: Alternate Quantity: Approximately 9 leaves, in the 10-15 cm section from the top of the branch. Average Length: 4.0 cm. Average Width: 2.5 cm. Shape of blade: Needle Apex: Needle-shaped Base: Elliptical Margin: Entire Texture of all surfaces: Hairy Aspect: Straight Color: Young foliage upper side: Yellow-green 146C RHS Young foliage under side: Yellow-green 146C RHS Mature foliage upper side: Green 137A RHS Mature foliage under side: Green 137A RHS Venation: Indistinguishable Petiole: Length: 0.7 cm. Diameter: 0.1 cm. Color: Green 137A RHS Texture: Hairy.
Flower:
   Natural flowering season.—Flowering rare in Adenanthos and not observed to date with new variety.
Other characteristics:
   Disease/pest resistance.—Neither resistance nor susceptibility to the normal diseases and pests of Adenanthos have been observed to date.
   Temperature tolerance.—From −2° C. to 35° C.
   Drought tolerance.—Extremely drought tolerant.

What is claimed is:

1. A new and distinct cultivar of Adenanthos plant named ‘AVIV’ as herein illustrated and described.
